(19) United States
(12) Patent Application Publication
(10) Pub. No.: US 2008/0033540 A1
(43) Pub. Date: Feb. 7, 2008

(54) Methods to prepare polymer blend implantable medical devices

(76) Inventors: Yunbing Wang, Sunnyvale, CA (US); David C. Gale, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/888,414

(22) Filed: Jul. 31, 2007

Related U.S. Application Data
(60) Provisional application No. 60/834,884, filed on Aug. 1, 2006.

(51) Int. Cl. A61F 2/06; C08L 53/00

(52) U.S. Cl. 623/1.49; 525/91; 623/1.15

(57) ABSTRACT

Methods and devices relating to polymer blend implantable medical devices are disclosed.

### CROSS REFERENCE TO RELATED APPLICATION

This application claims the benefit of and incorporates by reference U.S. Patent Application No. 60/834,884 which was filed on Aug. 1, 2006.

### BACKGROUND OF THE INVENTION

1. Field of the Invention

This invention relates to implantable medical devices and methods of fabricating implantable medical devices.

2. Description of the State of the Art

This invention relates to radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil. In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements of a pattern with respect to each other.

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug.

Furthermore, it may be desirable for a stent to be biodegradable. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers should be configured to completely erode only after the clinical need for them has ended.

Potential problems with polymeric stents include that they may have inadequate toughness and they may have a degradation rate that is slower than is desirable for certain treatments.

### SUMMARY OF THE INVENTION

Various embodiments of the present invention include a stent comprising a scaffolding fabricated from a polymer blend, the polymer blend comprising: a matrix polymer comprising PLLA; and a block copolymer comprising P(GA-co-CL)-b-PLLA dispersed within the PLLA, wherein the block copolymer is between 5-30 wt % of the polymer blend.

Further embodiments of the present invention include a stent comprising a scaffolding fabricated from a polymer blend, the polymer blend comprising: a matrix polymer comprising PLLA; an elastomeric polymer comprising P(GA-co-CL) forming a dispersed phase within the matrix polymer; and a compatibilizer block polymer comprising P(GA-co-CL)-b-PLLA dispersed within the blend, the compatibilizer block copolymer facilitating adhesion of the dispersed phase with the matrix polymer, the compatibilizer polymer being between about 0.2-5 wt % of the polymer blend.

Additional embodiments of the present invention include a stent comprising a scaffolding fabricated from a polymer blend, the polymer blend comprising: a matrix polymer comprising PLLA; and a block copolymer comprising P(GA-co-CL)-b-PLLA dispersed within the PLLA, wherein the block copolymer is between 5-30 wt % of the polymer blend.

Certain other embodiments of the present invention include a stent comprising a scaffolding fabricated from a polymer blend, the polymer blend comprising: a matrix polymer comprising a semi-crystalline polymer; and a block copolymer comprising an elastomeric bock and a semi-crystalline block dispersed within the matrix polymer, the elastomeric block forming a dispersed phase within the matrix polymer, wherein a content of the semicrystalline block is high enough that it facilitates adhesion between the dispersed phase and the matrix polymer, and wherein the content of the semi-crystalline block is low enough that the elastomeric block increases the toughness of the blend.

Additional embodiments of the present invention include a method of fabricating a stent comprising: radially expanding a polymer tube by applying an expansion pressure within the tube, wherein the polymer tube is composed of a matrix polymer comprising a semi-crystalline polymer and a block copolymer, the block copolymer including an elastomeric block and a semi-crystalline block dispersed within the matrix polymer, the block copolymer increasing the toughness of the polymer tube, wherein the pressure required to radially expand the polymer tube at a selected expansion rate and selected temperature is lower than a polymer tube made from a semi-crystalline polymer without dispersed high toughness polymer; and fabricating a stent from the expanded polymer tube.

### BRIEF DESCRIPTION OF THE DRAWINGS

FIG. 1A depicts a three-dimensional view of a stent.

FIG. 1B depicts a section of a structural element from the stent depicted in FIG. 1A.

FIG. 2 depicts a schematic close-up view of the section depicted in FIG. 1B.

FIG. 3 depicts a schematic close-up view of an interface between a discrete polymer phase and a continuous polymer phase.

FIG. 4 depicts a synthesis scheme of a modifier polymer.

FIGS. 5 and 6 depict an illustration of an exemplary blow molding process and apparatus.

### DETAILED DESCRIPTION OF THE INVENTION

Various embodiments of the present invention include an implantable medical device fabricated at least in part of a polymer blend composite including an elastomeric polymer phase dispersed within a matrix polymer.

As used herein, an “implantable medical device” includes, but is not limited to, self-expandable stents, balloon-expandable stents, stent-grafts, implantable cardiac pacemakers and defibrillators, leads and electrodes for the preceding, vascular grafts, grafts, artificial heart valves, and cerebrospinal fluid shunts.

An implantable medical device can be designed for the localized delivery of a therapeutic agent. A medicated implantable medical device may be constructed by coating the device or substrate with a coating material containing a therapeutic agent. The substrate of the device may also contain a therapeutic agent.

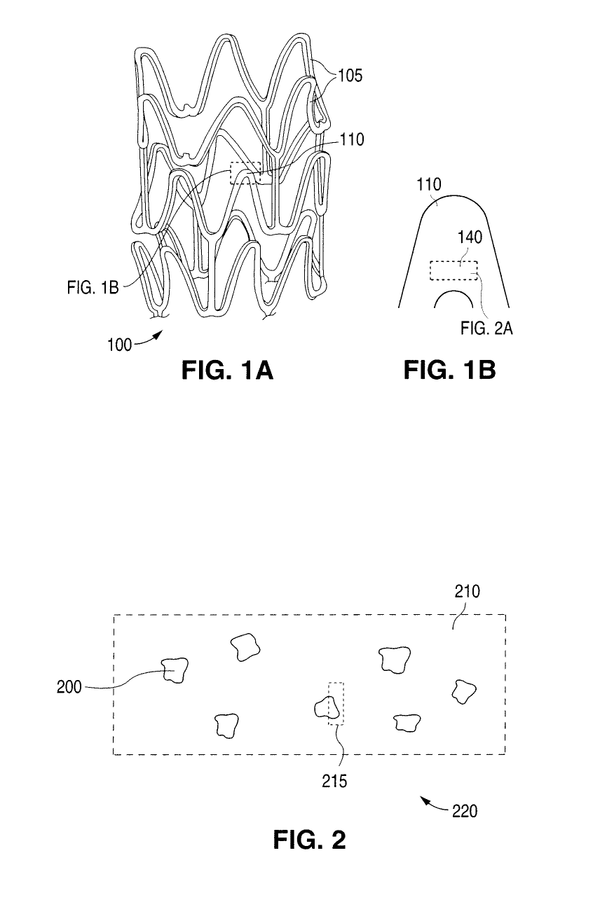

FIG. 1A depicts a three-dimensional view of a stent 100. In some embodiments, a stent may include a pattern or network of interconnecting structural elements 105. Stent 100 may be formed from a tube (not shown). The pattern of structural elements 110 can take on a variety of patterns. The structural pattern of the device can be of virtually any design. The embodiments disclosed herein are not limited to stents or to the stent pattern illustrated in FIG. 1A. The embodiments are easily applicable to other patterns and other devices. The variations in the structure of patterns are virtually unlimited. A stent such as stent 100 may be fabricated from a tube by forming a pattern with a technique such as laser cutting or chemical etching.

An implantable medical device can be made partially or completely from a biodegradable, bioabsorbable, or biostable polymer. A polymer for use in fabricating an implantable medical device can be biostable, bioabsorbable, biodegradable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed, and/or eliminated by the body. The processes of breaking down and absorption of the polymer can be caused by, for example, hydrolysis and metabolic processes.

Some polymers that may be suitable for implantable medical devices such as stents have potential shortcomings. For example, some crystalline or semi-crystalline polymers may be selected primarily on the basis of strength and stiffness at physiological conditions so that the stent substrate can provide adequate support for a lumen. Physiological conditions refer to conditions within a human patient including, but not limited, to body temperature. Such polymers may be glassy or have a Tg above body temperature making them stiff and strong at body temperature which is approximately 37° C. One such shortcoming of such crystalline or semi-crystalline polymers is that their toughness is lower than desired, in particular, for use in stent applications. For example, polymers such as poly(L-lactide) (PLLA) tend to be brittle under physiological conditions or conditions within a human body. These polymers can exhibit a brittle fracture mechanism in which there is little or no plastic deformation prior to failure. As a result, a stent fabricated from such polymers can have insufficient toughness for the range of use of a stent.

One way to increase fracture toughness of a low fracture toughness polymer under physiological conditions is to blend it with another polymer having a higher or relatively high fracture toughness under physiological conditions, such that the higher fracture toughness polymer is also immiscible and forms a discrete or dispersed phase from the low fracture toughness polymer. The discrete phase can absorb energy arising from stress imparted to a device made from the blend to increase the fracture toughness of the device. To ensure good energy transfer between interfaces of the phases, it is important that there be sufficient bonding or adhesion between the phases. See, Y. Wang, etc. Journal of Polymer Science Part A: Polymer Chemistry, 39, 2001, 2755-2766.

Another such shortcoming is that such biodegradable polymers may have a degradation rate that is slower than desired for certain stent treatments. As a result, the degradation time of a stent made from such polymers can be longer than desired. For example, a stent made from poly(L-lactide) (PLLA) can have a degradation time of between about two and three years. In some treatment situations, a degradation time of less than a year may be desirable, for example, between four and eight months.

The degradation of a hydrolytically degradable polymer follows a sequence including water penetration into the polymer followed by hydrolysis of bonds in the polymer. Thus, the degradation of a polymer can be influenced by its affinity for water and the diffusion rate of water through the polymer. A hydrophobic polymer has a low affinity for water which results in a relatively low water penetration. In addition, the diffusion rate of water through crystalline regions of a polymer is lower than amorphous regions. Thus, as either the affinity of a polymer for water decreases or the crystallinity increases, water penetration and water content of a polymer decreases.

Certain embodiments of an implantable medical device can include an implantable medical device fabricated at least in part of a polymer blend or polymer-polymer composite. In some embodiments, the device can be a stent. In such embodiments, one or more structural elements or struts of a stent can be fabricated from the composite. In other such embodiments, the body, scaffolding, or substrate of a stent can be made from the composite.

A stent body, scaffolding, or substrate can refer to a stent structure with an outer surface to which no coating or layer of material different from that of which the structure is manufactured has not yet been applied. If the body is manufactured by a coating process, the stent body can refer to a state prior to application of optional additional coating layers of different material. By “outer surface” is meant any surface however spatially oriented that is in contact with bodily tissue or fluids. A stent body, scaffolding, or substrate can refer to a stent structure formed by laser cutting a pattern into a tube or a sheet that has been rolled into a cylindrical shape.

In some embodiments, a majority, substantially all, or all of the stent body, scaffolding, or substrate can be made from the composite. Substantially all of the body can refer to greater than 90%, 95%, or greater than 99% of the body.

In some embodiments, the polymer blend can include a mixture of a matrix polymer with a modifier copolymer, the matrix polymer being a majority of the polymer blend, where majority means greater than 50%. The modifier polymer can be a block copolymer with the matrix polymer being immiscible with at least some of the blocks or segments of the modifier polymer. Since at least some of the blocks or segments of the modifier polymer are immiscible with the matrix polymer, the composite includes a continuous matrix polymer phase and a discrete or modifier polymer phase dispersed within the continuous phase.

Furthermore, the continuous phase can include the matrix polymer and the discrete phase can include the immiscible blocks of the modifier polymer. In one embodiment, the matrix polymer has a relatively low fracture toughness at physiological conditions and the modifier polymer has a higher fracture toughness at physiological conditions. The modifier polymer or discrete phase tends to increase the toughness of the matrix polymer, and thus the composite.

In some embodiments, the modifier polymer can have discrete phase segments and anchor segments. The discrete phase segments are immiscible with the matrix polymer so that the discrete phase segments are in the discrete phase. Additionally, the discrete phase is a high toughness polymer than increases the fracture toughness of the composite. The anchor segments are miscible with the matrix polymer so that the anchor segments at least partially phase separate from the discrete phase into the continuous phase.

FIG. 1B depicts a section of a segment 110 of strut 105 from the stent depicted in FIG. 1A. FIG. 2 depicts a microscopic section 220 of a portion 140 of segment 110 of the strut as depicted in FIG. 1B. Portion 140 includes a discrete or dispersed phase 200 within a continuous phase 210.

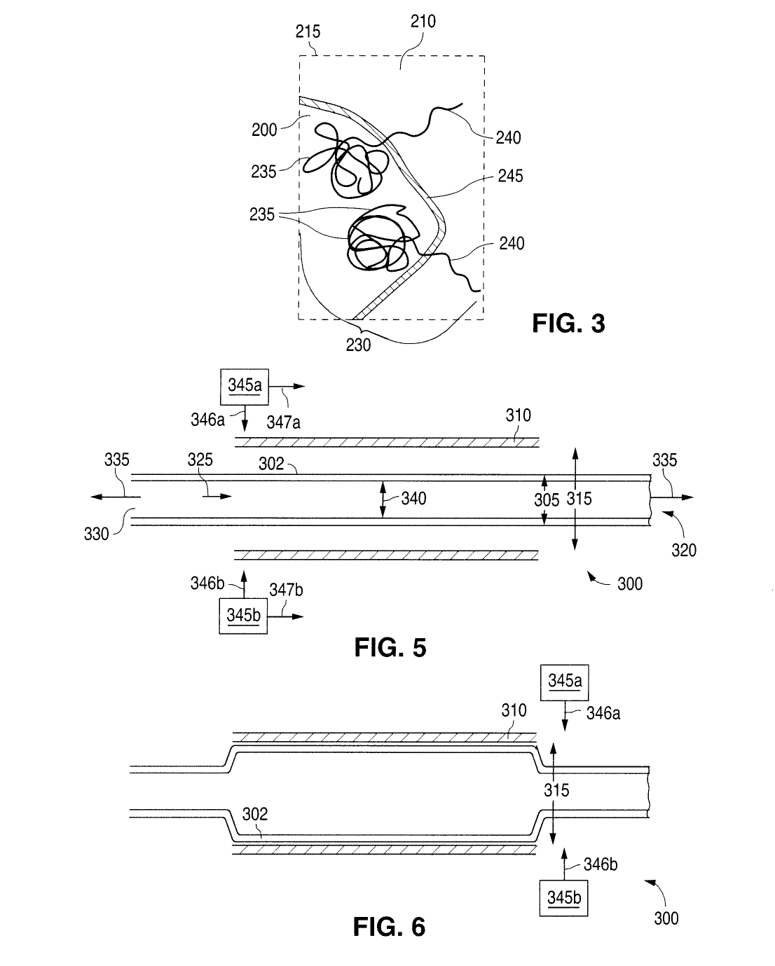

FIG. 3 depicts a schematic close-up view of section 250 including an interface between discrete phase 200 and continuous polymer phase 210. A modifier polymer 230 is shown to have discrete phase segments 235 and anchor segments 240. Line 245 is meant to delineate the boundary between discrete phase 200 and continuous phase 210. Anchor segments 240 are shown to be phase separated from discrete phase 200 into continuous phase 210.

It is believed that when a device is placed under stress, the discrete phase tends to absorb energy when a fracture starts to propagate through a structural element. Crack propagation through the continuous phase may then be reduced or inhibited. As a result, fracture toughness of the blend of the matrix polymer and the modifier polymer, and thus the structural element tends to be increased. Furthermore, the anchor segments tend to increase the adhesion between the discrete phase and the continuous phase. Thus, the anchor segments facilitate energy transfer between interfaces of the phases.

Additionally, the molecular weight or content of the anchor blocks in the modifier polymer should be high enough to sufficiently increase the interfacial adhesion between the discrete phase and continuous phase. As the molecular weight or content of the anchor block decreases, the degree of adhesion decreases. Furthermore, as the molecular weight or content of the anchor blocks increases, the toughness and degradation rate of the composite can decrease. Thus, there are upper and lower limits of the molecular weight or content of the anchor blocks that provide desired toughness and degradation rate of the composite.

In some embodiments, the discrete phase segments of the modifier polymer can include units or functional groups that form polymers that have a higher fracture toughness than a matrix polymer such as PLLA. The discrete phase segments can form a discrete phase that is more flexible and has a lower modulus than the matrix polymer of the continuous phase. In some embodiments, the discrete phase segments of the modifier polymer are a rubbery or elastomeric polymer. An “elastomer” or “rubbery” polymer refers to a polymer which can resist and recover from deformation produced by force, as in natural rubber. In one embodiment, elastomers or rubbery polymers can be stretched repeatedly to at least twice their original length and, immediately upon release of the stress, return with force to their approximate original length. In another embodiment, elastomers or rubbery polymers are substantially or completely amorphous polymers that are above their Tg's. In an embodiment, the discrete phase segments of the modifier polymer have a Tg below body temperature.

Biodegradable polymers having a relatively high fracture toughness include, but are not limited to, polycaprolactone (PCL) and poly(trimethylene carbonate) (PTMC), polydioxanone (PDO), poly(4-hydroxy butyrate) (PHB), poly(butylene succinate) (PBS). PCL and PTMC are known to be immiscible in PLLA. Thus, some embodiments of the discrete phase segments of the modifier polymer can include caprolactone (CL) and/or tetramethyl carbonate (TMC) monomers. The fraction of the CL and/or TMC monomers can be high enough that the discrete phase segments are immiscible in the PLLA.

Furthermore, a matrix polymer, such as PLLA, can have a degradation rate that is slower than desired for certain stent treatments. Thus, in additional embodiments the modifier polymer can include hydrolytically degradable units or functional groups that provide desired degradation characteristics. In some embodiment, the discrete phase segments of the modifier polymer can include functional groups or monomers that increase water penetration and content in the discrete phase and in the continuous phase. In particular, the discrete phase segments can include monomers that have a higher affinity for water and/or are more hydrolytically active than the matrix polymer. For example, the discrete phase segments can include glycolide (GA) monomers which are faster degrading than L-lactide monomers.

In other embodiments, the discrete phase segments can include units that increase the fracture toughness of the polymer blend and units that increase the degradation rate of the polymer blend. In an embodiment, the discrete phase segments can include both CL and GA monomers. In particular, the discrete phase segments can be poly(glycolide-co-ε-caprolactone) (P(GA-co-CL)). P(GA-co-CL) discrete phase segments can have alternating or random GA and CL monomers. The faster degrading GA monomers can increase the degradation rate of the polymer blend by increasing the equilibrium water content and penetration into the structural element. The acidic and hydrophilic degradation products of the GA segments also act to increase the degradation rate of the polymer blend.

In some embodiments, the flexibility and degradation rate of the discrete phase segments can be adjusted by the ratio of fast degrading and high toughness units. As the ratio of CL, for example, increases in P(GA-co-CL) segments, the polymer becomes more flexible and tougher. The Tg of the discrete phase segments can be tuned to a desired value by adjusting the ratio of component monomers. For example, the Tg of the discrete phase may be engineered to be less than a body temperature to provide a more flexible discrete phase under physiological conditions. Additionally, the degradation rate of the discrete phase segments, and thus the blend, can be increased by increasing the fraction of GA in the discrete phase segments. In exemplary embodiments, the P(GA-co-CL) segments can have greater than 1 wt %, 5 wt %, 20 wt %, 50 wt %, 70 wt %, 80 wt %, or 90 wt % GA monomer.

In one embodiment, the modifier polymer can include P(GA-co-CL)-b-PLLA. The discrete phase segment is P(GA-co-CL) and the anchor segment is PLLA. In a binary polymer blend of a modifier polymer of P(GA-co-CL)-b-PLLA with a matrix polymer of PLLA, the PLLA anchor segment of the modifier polymer can phase separate into the PLLA matrix of the continuous phase. The PLLA anchor segment can bind the discrete phase with the continuous phase, facilitating the increase in the fracture toughness of the polymer blend.

In exemplary embodiments, the polymer blend or composite can include about 1-40 wt %, or more narrowly 5-30 wt % of a modifier polymer and about 75-95 wt % of matrix polymer.

In exemplary embodiments, the LLA content in P(GA-co-CL)-b-PLLA is less than than 20 wt % or the molecular weight is less than 50 kg/mol. It has been observed that to provide an adequate degree of adhesion between the discrete and continuous phases, the content of the LLA in P(GA-co-CL)-b-PLLA copolymer should be greater than about 20 wt % or the molecular weight should be greater than 50 kg/mol. It has also been observed that an adequate toughness and degradation rate of the composite it obtained for an anchor block content less than about 50 wt %.

In further embodiments, the matrix polymer can be a copolymer. In some embodiments, a matrix copolymer can be composed of functional groups with different properties. In an embodiment, the copolymer can include functional groups selected to increase the degradation rate of the copolymer. Such a functional group can have a greater affinity for water or be more hydrolytically active than other functional groups of the copolymer.

In an exemplary embodiment, the matrix copolymer can be poly(L-lactide-co-glycolide) (LPLG). Increasing the content of GA can increase the degradation rate of the LPLG since GA is more hydrolytically active than LLA. The weight percent of the GA in the copolymer can be at least about 1%, 5%, 10%, 15%, 30%, 40%, or at least about 50%. In certain exemplary embodiments, the weight percent of the GA group can be adjusted so that the degradation time of a stent scaffolding can be less than 18 months, 12 months, 8 months, 5 months, 3 months, or more narrowly, less than 3 month.

Additionally, the anchor segment of the modifier polymer can be selected so that the anchor segment is miscible with the matrix copolymer. In one embodiment, the anchor segment can have the same composition as the matrix copolymer. In another embodiment, the anchor segment can have a composition different from the matrix copolymer, but close enough so that the anchor segment is miscible with the matrix polymer. In another embodiment, the anchor segment can have composition different from the matrix polymer with the anchor segments being miscible with the matrix polymer.

Some embodiments can include a matrix polymer of PLLA and anchor blocks that include 100% L-lactide units or both L-lactide and GA units. Other embodiments can include a matrix polymer of LPLG and anchor blocks that include 100% L-lactide units or both L-lactide and GA units.

In some embodiments, a blend for fabricating an implantable medical device can be a ternary blend of the matrix polymer, a modifier polymer with discrete phase segments and an anchor block, and a discrete phase copolymer composed of discrete phase segments of the modifier polymer. The matrix polymer can form a continuous phase and the discrete phase copolymer can form a discrete phase within the continuous phase. The modifier polymer may act as a compatibilizer for the matrix polymer and the discrete phase copolymer by facilitating adhesion between the discrete and continuous phases. In general, a “compatibilizer” refers to an interfacial agent that modifies the properties of an immiscible polymer blend which facilitates formation of uniform blend, and increases interfacial adhesion between the phases. Compatibilization refers to the process of modification of the interfacial properties in an immiscible polymer blend that results in formation of interphases (region of concentration gradient between phases) and stabilization of the morphology. In one embodiment, the copolymer with discrete phase segments is a majority of the discrete phase.

In an exemplary embodiment, a ternary blend can include PLLA as the matrix polymer; P(GA-co-CL) copolymer; and P(GA-co-CL)-b-PLLA. In such embodiments, P(GA-co-CL) copolymer is in the discrete phase along with P(GA-co-CL) segments of the modifier polymer. PLLA of the modifier polymer phase separates into the PLLA continuous phase. In another exemplary embodiment, a ternary blend can include LGLG as the matrix polymer; P(GA-co-CL) copolymer; and P(GA-co-CL)-b-LPLG and/or LPLG-b-P(GA-co-CL) as the modifier polymer. In such embodiments, P(GA-co-CL) copolymer is in the discrete phase along with P(GA-co-CL) segments of the modifier polymer. LPLG of the modifier polymer phase separates into the LPLG continuous phase. In exemplary embodiments, a ternary polymer blend can include about 1-40 wt %, or more narrowly, 5-30 wt % of a P(GA-co-CL); about 1-5% wt % of P(GA-co-CL)-b-PLLA, and about 75-95 wt % of matrix polymer.

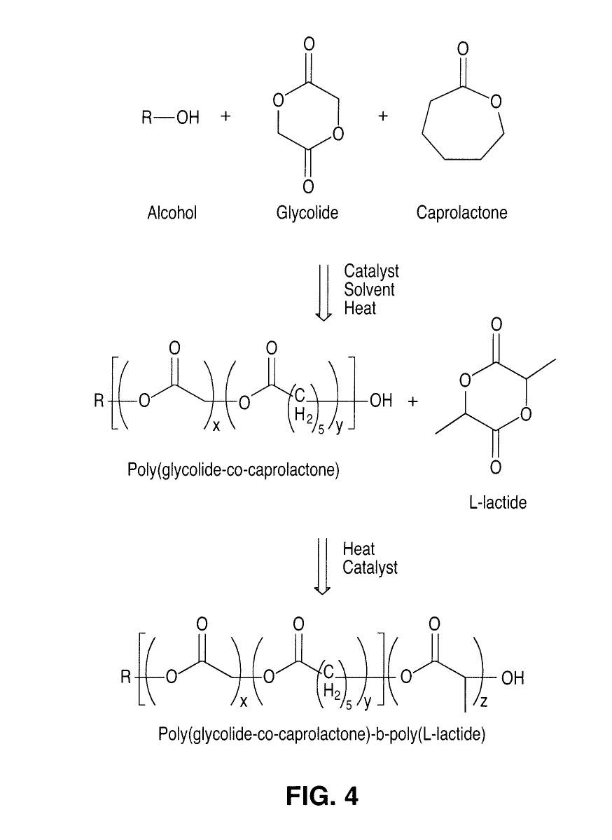

In some embodiments, a modifier polymer, such as P(GA-co-CL)-b-PLLA or P(GA-co-CL)-b-LPLG, can be formed by solution-based polymerization. Other methods used to form the modifier polymers are also possible, such as, without limitation, melt phase polymerization. In solution-based polymerization, all the reactive components involved in the polymerization reaction are dissolved in a solvent. To prepare P(GA-co-CL)-b-PLLA copolymer, P(GA-co-CL) may be prepared first by solution polymerization and then employed as a macro-initiator to initiate the polymerization of L-lactide monomers to form the PLLA segment, as illustrated in FIG. 4. Specifically, P(GA-co-CL) segments are formed first by mixing GA monomers and CL monomers with a solvent to form a solution. In the solution, the GA and CL monomers react to form P(GA-co-CL). L-lactide monomers can then be added to the solution or another solution containing the formed P(GA-co-CL). The L-lactide monomers react with P(GA-co-CL) to form P(GA-co-CL)-b-PLLA.

In one embodiment, the L-lactide monomers react in the same solution as the solution used to form P(GA-co-CL). Alternatively, the L-lactide monomers can react in a solution having a different solvent than the solution for forming P(GA-co-CL). The solvent(s) for forming the PLLA anchor segment can be selected so that the P(GA-co-CL) copolymer is soluble in the solvent(s) so that the copolymer can further copolymerize with L-lactide monomers.

In other embodiments, P(GA-co-CL)-b-PLLA can be formed by reacting P(GA-co-CL) copolymer swollen with a solvent with L-lactide monomers. One of skill in the art can select a solvent that swells but does not dissolve P(GA-co-CL). P(GA-co-CL) copolymer is swollen by a solvent after it is formed so that the P(GA-co-CL) copolymer can react with added L-lactide monomers.

In another embodiment, the synthesis of the PLLA-b-P(GA-co-CL) copolymer can be performed by first synthesizing the PLLA block. The L-lactide monomers can be mixed with a solvent to form a solution. GA monomers and CL monomers are added to a solution containing the PLLA that is formed to form PLLA-b-P(GA-co-CL) copolymer. The solution can be the same solution used to form the PLLA or a solution containing a different solvent.

In one embodiment, the solvent for use in synthesizing the copolymer is devoid of alcohol functional groups. Such alcoholic groups may act as initiators for chain growth in the polymer. Solvents used to synthesize the copolymer include, but are not limited to, chloroform, toluene, xylene, and cyclohexane. Initiators to facilitate the synthesis of the copolymer include, but are not limited to, dodecanol, ethanol, ethylene glycol, and polyethylene glycol. Catalysts used to facilitate the synthesis of the copolymer include, but are not limited to, stannous octoate and stannous trifluoromethane sulfonate.

In some embodiments, the polymer blend or composite can be formed by melt blending. In melt blending the bioceramic particles are mixed with a polymer melt. The particles can be mixed with the polymer melt using extrusion or batch processing. A composite of the polymer blend and bioceramic particles can be extruded to form a polymer construct, such as a tube. A stent can then be fabricated from the tube.

Further embodiments of the method include conveying the composite mixture into an extruder. The composite mixture may be extruded at a temperature above the melting temperature of the polymers in the composite mixture and less than the melting temperature of the bioceramic particles. In some embodiments, the dried composite mixture may be broken into small pieces by, for example, chopping or grinding. Extruding smaller pieces of the composite mixture may lead to a more uniform distribution of the nanoparticles during the extrusion process.

The extruded composite mixture may then be formed into a polymer construct, such as a tube or sheet which can be rolled or bonded to form a tube. A medical device may then be fabricated from the construct. For example, a stent can be fabricated from a tube by laser machining a pattern in to the tube. In another embodiment, a polymer construct may be formed from the composite mixture using an injection molding apparatus.

As indicated above, it is important for a stent to have high radial strength so that once it is deployed from the crimped state, it can support a lumen. In general, deforming a polymer construct can strengthen the polymer of the construct along an axis of deformation. In some embodiments of fabricating a stent from a polymer tube, the polymer tube can be radially expanded to increase the radial strength of the tube. The stent can then be fabricated from the polymer tube in its expanded state.

In certain embodiments, a polymeric tube formed from the polymeric blend can be radially expanded from an extruded diameter to a target diameter prior to forming a pattern in the tube. In such embodiments, the tube can be expanded, for example, 300% to 700% of its original inside diameter (ID) to increase the radial strength of tubing. In some embodiments, the tube can be radially expanded using blow molding. In a blow molding process, a polymer tube is disposed within a mold having an outside diameter that is the target diameter of an expanded tube. The mold is typically made of a material that allows easy release of the tube once expanded, for example, glass. To expand the tube, the pressure inside the tube is increased, typically by blowing a gas into the tube disposed within the mold.

Additionally, the tube is heated to a temperature that facilitates expansion of the tube. As the temperature increases, the polymer tube more readily deforms. For example, the tube can be heated to a temperature above the Tg of the polymeric tube. In some embodiments, the tube can be heated by a heating nozzle that is positioned adjacent to the mold. The nozzle directs a heated gas at one or more positions around the circumference of the tube. The heated gas flows around and heats the mold and the tube around the circumference. The polymer tube radially expands due the increased pressure and the heating.

FIGS. 5 and 6 depict an illustration of an exemplary blow molding process and apparatus. FIG. 5 depicts an axial cross-sectional view of a blow molding apparatus 300 with a polymer tube 302 positioned within a tubular mold 310. Polymer tube 302 has an initial outside diameter 305. Mold 310 limits the radial deformation of polymer tube 302 to an outside diameter 315 of mold 310. A nozzle with fluid ports 345a and 345b directs a heated gas, as shown by arrows 346a and 346b, at opposite sides of mold 310.

In a blow molding process illustrated in FIGS. 5-6, a fluid (conventionally a gas such as air, nitrogen, oxygen, argon, etc.) may be conveyed, as indicated by an arrow 325, into an open proximal end 330 to increase the pressure within polymer tube 302. Polymer tube 302 is closed at a distal end 320, but may be open in subsequent manufacturing steps. Distal end 320 may be open in subsequent manufacturing steps. Optionally, a tensile force 335 may be applied at proximal end 330 or distal end 320, or both. A nozzle with fluid ports 345a and 345b is translated axially as shown by arrows 347a and 347b, respectively. As indicated above, a heated stream of gas, as shown by arrows 346a and 346b, is directed on mold 310 and flows around the circumference of mold 310 to heat regions not directly impacted by the heated gas stream from ports 345a and 345b. Embodiments of the present invention are not limited to the manner of heating with a nozzle as illustrated in FIGS. 5 and 6.

Polymer tube 302 radially expands, as shown by arrow 340 in FIG. 5, as the nozzle translates axially. The radial deformation is facilitated by the heating by the nozzle and the increase in pressure inside of polymer tube 302. The temperature of the polymer tube can be heated to a temperature above the Tg of the polymer of the tube. FIG. 6 depicts polymer tube 302 in a deformed state with an outside diameter 315 within mold 310.

It has been observed that a polymer blend tube is tougher and more flexible than a polymer tube composed of the semi-crystalline matrix polymer without a blended elastomeric polymer, such as embodiments of the modifier polymer described above. Thus, in some embodiments, the processing conditions for expansion of a polymer blend tube may be modified as compared to a polymer tube made from a matrix polymer to account for the difference in toughness and flexibility. Since heating a polymer tube facilitates expansion, it is expected that a lower expansion temperature may be employed. Alternatively, or additionally, a lower expansion pressure can be used due to the higher toughness and flexibility of the polymer blend tube.

Thus, the pressure required to expand a polymer blend tube at a selected expansion rate tends to be lower than a tube made from a semi-crystalline polymer without dispersed high toughness polymer. In some embodiments, the pressure to expand a polymer blend tube can be at least 10%, 20%, 30%, 40% or 50% less than a pure semi-crystalline polymer tube. Selecting the expansion pressure is important since the properties of the expanded tube can be influenced by the expansion pressure. It is believed that the expansion rate can influence properties of the tubing. The expansion rate tends to increase with the expansion pressure. Additionally, it is desirable to select an expansion pressure that avoids damaging the tube.

In an exemplary embodiment, a pure PLLA tubing, can be expanded in a temperature range 80-140° C. For a selected expansion rate, the expansion pressure can be about 130-180 psi. For a PLLA blend tubing expansion having the same expansion temperature range and expansion rate, the expansion pressure can be 90-120 psi.

Representative examples of polymers that may be used to fabricate an implantable medical device include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide); poly(D,L-lactide), poly(caprolactone), poly(trimethylene carbonate), polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

Additional representative examples of polymers that may be especially well suited for use in fabricating an implantable medical device according to the methods disclosed herein include ethylene vinyl alcohol copolymer (commonly known by the generic name EVOH or by the trade name EVAL), poly(butyl methacrylate), poly(vinylidene fluoride-co-hexafluororpropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol. For the purposes of the present invention, the following terms and definitions apply:

As indicated above, an implantable medical device such as a stent can be medicated by incorporating an active agent in a coating over the device or within the substrate of the device. In some embodiments, the ions released from bioceramics can have an additive therapeutic and/or a synergistic therapeutic effect to the active agent. For example, ions can be used in conjunction with anti-proliferative and/or anti-inflammatory agents.

For the purposes of the present invention, the following terms and definitions apply:

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

“Stress” refers to force per unit area, as in the force acting through a small area within a plane. Stress can be divided into components, normal and parallel to the plane, called normal stress and shear stress, respectively. True stress denotes the stress where force and area are measured at the same time. Conventional stress, as applied to tension and compression tests, is force divided by the original gauge length.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, amount of deformation, and the strain rate or rate of deformation. For example, below its Tg, a polymer tends to be brittle with a high modulus. As the temperature of a polymer is increased from below to above its Tg, its modulus decreases.

“Strain” refers to the amount of elongation or compression that occurs in a material at a given stress or load.

“Elongation” may be defined as the increase in length in a material which occurs when subjected to stress. It is typically expressed as a percentage of the original length.

“Toughness” is the amount of energy absorbed prior to fracture, or equivalently, the amount of work required to fracture a material. One measure of toughness is the area under a stress-strain curve from zero strain to the strain at fracture. Thus, a brittle material tends to have a relatively low toughness.

“Solvent” is defined as a substance capable of dissolving or dispersing one or more other substances or capable of at least partially dissolving or dispersing the substance(s) to form a uniformly dispersed solution at the molecular- or ionic-size level at a selected temperature and pressure. The solvent should be capable of dissolving at least 0.1 mg of the polymer in 1 ml of the solvent, and more narrowly 0.5 mg in 1 ml at the selected temperature and pressure, for example, ambient temperature and ambient pressure.

### EXAMPLES

The examples and experimental data set forth below are for illustrative purposes only and are in no way meant to limit the invention. The following examples are given to aid in understanding the invention, but it is to be understood that the invention is not limited to the particular materials or procedures of examples.

The Examples below are provided by way of illustration only and not by way of limitation. The prophetic and actual Examples illustrate formation of P(GA-co-CL)-b-PLLA copolymer, formation of a blend of a block copolymer and matrix polymer, formation of a stent from the blend, and property evaluation of formed stents. This block copolymer includes two blocks: the P(GA-co-CL) copolymer and PLLA anchor blocks. The parameters and data are not to be construed to limit the scope of the embodiments of the invention.

### Example 1

### Synthesis of P(GA-co-CL)-b-PLLA Polymer Through Solution Polymerization

P(GA-co-CL)-b-PLLA copolymer was synthesized by forming the P(GA-co-CL) segments first. Then, P(GA-co-CL) is used to initiate polymerization of LLA. FIG. 4 illustrates the synthesis of P(GA-co-CL)-b-PLLA copolymer. FIG. 4 shows that GA and CL monomers are combined in the presence of an alcohol initiator, catalyst, and solvent to form P(GA-co-CL). LLA is then added to the mixture to form the P(GA-co-CL) copolymer.

Generally, toluene, xylene, or cyclohexane can be used as the solvent. It has been found that toluene is a better solvent for the P(GA-co-CL)-b-PLLA block copolymer and the PLLA block than other solvents. The reactants can be dissolved in the solvent during the early stages of polymerization. The solvent can be removed at higher temperature to increase polymerization rate. Initiators can include dodecanol and ethanol.

Catalysts can include stannous octoate and/or stannous trifluoromethane sulfonate.

The following steps describe a polymerization procedure that was used to form P(GA-co-CL)-b-PLLA:

Step 1: A 2-L reaction kettle with mechanical stirring rod was placed into a glove box which was filled with nitrogen.

Step 2: 300 ml toluene, 130 g GA, 70 g CL, 0.11 mL dodecanol, and 0.56 mL stannous octoate were added to the reaction kettle. The temperature was increased to between 100-120° C.

Step 3: After 70 hours, 200 g LLA was then added to the reaction kettle.

Step 4: After 65 hours, P(GA-co-CL)-b-PLLA product was precipitated by adding the reaction solution into methanol. The product was filtered and dried in vacuum overnight.

Based on NMR analysis, neither caprolactone nor glycolide monomer existed before LLA was added and nearly 95% LLA monomer had been converted into polymer chain.

The GPC results showed that no PLLA homopolymer was formed and PGA-co-CL-b-PLLA copolymer had been successfully synthesized.

### Example 2

### PLLA Blend Preparation and Tubing Preparation

The PLLA/copolymer blend can be prepared by either solution blending or melting blending. In the case of solution blending, the PLLA and copolymer is dissolved in a co-solvent and then precipitated out of non-solvent. For melt blending, the copolymer is first cut into pieces and then blended with PLLA in single or twin screw extruder. The tubing with a designed inner and outer diameter is thus obtained through a puller after extrusion. In practice, PLLA can be mixed with 5% to 30% PGA-co-CL-b-PLLA copolymer to form a binary blend. PLLA can also be mixed with 5% to 30% PGA-co-CL copolymer to form a ternary blend using 1%-5% PGA-co-CL-b-PLLA copolymer as compatibilizer. The following example illustrates the preparation of PLLA/modifier polymer blend and tube:

Step 1: Cut synthesized PGA-co-PCL-b-PLLA copolymer into small pieces with blender.

Step 2: Extrude PLLA/PGA-co-PCL-b-PLLA binary blend (100:10) through single screw extruder at 430° F. with puller speed at 20 rpm. Final inner diameter (ID) of tubing was 0.021 in and outer diameter (OD) was 0.072 in.

### Example 3

### Stent Preparation Through Radial Expansion and Laser Cutting

PLLA/PGA-co-PCL-b-PLLA blend tubing is expanded 300% to 700% of its original ID to increase the radial strength of tubing. The temperature ranges during radial expansion can be from 80° C. to 140° C., the pressure of nitrogen to expand tubing can be 70 to 120 psi. The stent is cut by femto-second laser. The stent is crimped at 25-60° C. The stent is sterilized by electron beam, ethylene oxide, UV, or gamma ray.

The following example illustrates the preparation of PLLA/PGA-co-PCL-b-PLLA stent:

Step 1: Expanded the extruded tubing radially to 500% of its original ID.

Step 2: Cut stent by femto-second laser using laser power ranging from 115 to 150 mW.

Step 3: Crimped the expanded tubing at 30° C. to 0.053 in.

Step 4: Sterilized stent by electron beam at 20° C. using 16-33 KGray dose.

### Example 4

### Property Evaluation of Stent Made from PLLA/Copolymer Blend

For fracture resistance comparison, PLLA Stents and PLLA blend stents are deployed to certain size. Both stents are stored at 40° C. for accelerated shelf life testing

Both stents are stored in saline buffer solution for degradation testing.

The following example illustrates the property evaluation of PLLA/PGA-co-PCL-b-PLLA blend stent:

Step 1: 10 stents made in Example 3 were deployed to 4.0 mm at 37° C. and no broken struts were observed. Stents made from pure PLLA were broken after being deployed to 4.0 mm.

Step 2: 5 stents made in Example 3 were stored at 40° C. for 16 h and then deployed to 4.0 mm. No broken struts were found. Most stents made from pure PLLA were broken after being deployed to only 3.0 mm.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A stent comprising a scaffolding fabricated from a polymer blend, the polymer blend comprising:
a matrix polymer comprising PLLA; and
a block copolymer comprising P(GA-co-CL)-b-PLLA dispersed within the PLLA, wherein the block copolymer is between 5-30 wt % of the polymer blend.

2. The stent of claim 1, wherein the PLLA is greater than 95 wt % of the matrix polymer.

3. The stent of claim 1, wherein the P(GA-co-CL) blocks of the block copolymer form a dispersed phase within the matrix polymer.

4. A stent comprising a scaffolding fabricated from a polymer blend, the polymer blend comprising:
a matrix polymer comprising PLLA;
an elastomeric polymer comprising P(GA-co-CL) forming a dispersed phase within the matrix polymer; and
a compatibilizer block polymer comprising P(GA-co-CL)-b-PLLA dispersed within the blend, the compatibilizer block copolymer facilitating adhesion of the dispersed phase with the matrix polymer, the compatibilizer polymer being between about 0.2-5 wt % of the polymer blend.

5. The stent of claim 4, wherein the PLLA is greater than 95 wt % of the matrix polymer.

6. A stent comprising a scaffolding fabricated from a polymer blend, the polymer blend comprising:
a matrix polymer comprising PLLA; and
a block copolymer comprising P(GA-co-CL)-b-PLLA dispersed within the PLLA, wherein the block copolymer is between 5-30 wt % of the polymer blend.

7. The stent of claim 6, wherein the PLLA is greater than 95 wt % of the matrix polymer.

8. A stent comprising a scaffolding fabricated from a polymer blend, the polymer blend comprising:
a matrix polymer comprising a semi-crystalline polymer; and
a block copolymer comprising an elastomeric block and a semi-crystalline block dispersed within the matrix polymer, the elastomeric block forming a dispersed phase within the matrix polymer, wherein a content of the semicrystalline block is high enough that it facilitates adhesion between the dispersed phase and the matrix polymer, and wherein the content of the semi-crystalline block is low enough that the elastomeric block increases the toughness of the blend.

9. The method of claim 8, wherein the matrix polymer comprises PLLA and the block copolymer comprises P(GA-co-CL)-b-PLLA.

10. The method of claim 9, wherein a content of the PLLA block is at least 20 wt % of the P(GA-co-CL)-b-PLLA.

11. The method of claim 9, wherein a content of the PLLA block is less than 50 wt % of the P(GA-co-CL)-b-PLLA.

12. A method of fabricating a stent comprising:
radially expanding a polymer tube by applying an expansion pressure within the tube, wherein the polymer tube is composed of a matrix polymer comprising a semi-crystalline polymer and a block copolymer, the block copolymer including an elastomeric block and a semi-crystalline block dispersed within the matrix polymer, the block copolymer increasing the toughness of the polymer tube, wherein the pressure required to radially expand the polymer tube at a selected expansion rate and selected temperature is lower than a polymer tube made from a semi-crystalline polymer without dispersed high toughness polymer; and
fabricating a stent from the expanded polymer tube.

13. The method of claim 12, wherein the matrix polymer comprises PLLA and the block copolymer comprises P(GA-co-CL)-b-PLLA.

14. The method of claim 13, wherein the selected temperature between 80-140° C., and wherein the pressure to expand the polymer tube composed of P(GA-co-CL)-b-PLLA dispersed in PLLA is between 90-120 psi and the pressure to expand a polymer tube composed of PLLA is between 130-180 psi.
